(19) United States
(12) Patent Application Publication
(10) Pub. No.: US 2008/0009863 A1
(43) Pub. Date: Jan. 10, 2008

(54) PEDICLE SCREW DISTRACTOR AND ASSOCIATED METHOD OF USE

(75) Inventors: Meredith L. Bond, Bloomington, MN (US); Jack A. Dant, St. Paul, MN (US)

Correspondence Address: WOOD, HERRON & EVANS (ZIMMER SPINE), 2700 CAREW TOWER, 441 VINE STREET, CINCINNATI, OH, 45202, US

(73) Assignee: ZIMMER SPINE, INC., Minneapolis, MN (US)

(21) Appl. No.: 11/426,030

(22) Filed: Jun. 23, 2006

(51) Int. Cl. A61F 2/30

(52) U.S. Cl. 606/61

(57) ABSTRACT

A distractor for pedicle screws aids surgeons in counteracting the compressive force on a spacer between the pedicle screws and assists in the process of seating the spacer as part of a spinal dynamic stabilization system. The distractor cooperates with existing instrumentation to distract the pedicle screw heads for simple, efficient and accurate seating of the spacer. The pedicle screw distractor includes a head specifically designed and adapted to engage one of the pedicle screws seated in the patient's spine. A lever extends from the head of the pedicle screw distractor and is joined to a handle of the instrument to form a fulcrum arrangement. A saddle is provided at the fulcrum and is adapted to receive and seat the instrument on the shaft or body of another instrument mounted to an adjacent pedicle screw. The pedicle screw distractor when seated against the accompanying instrument and the installed pedicle screws provides for a convenient and simple distraction process by squeezing the handle toward the companion instrument thereby temporarily distracting the pedicle screws and providing access between the adjacent pedicle screws to seat the spacer of the spinal stabilization system there between.

### TECHNICAL FIELD

This invention relates generally to spinal fixation devices and more specifically relates to a system and associated method for distracting pedicle screws as part of the installation of spinal fixation devices.

### BACKGROUND

The human spinal column is a highly complex system of bones and connective tissues that provides support for the body and protects the delicate spinal cord and nerves. The spinal column includes a series of vertebrae stacked one atop the other. Each vertebral body includes a relatively strong cortical bone portion forming the outside surface of the body and a relatively weak cancellous bone portion forming the center of the body. An intervertebral disc is situated between each vertebral body that provides for cushioning and dampening of compressive forces applied to the spinal column. The vertebral canal containing the delicate spinal cords and nerves is located just posterior to the vertebral bodies.

Various types of spinal column disorders include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine). Other disorders are caused by abnormalities, disease or trauma, such as ruptured or slipped discs, degenerative disc disease (DDD), fractured vertebra, and the like. Patients that suffer from such conditions usually experience extreme and debilitating pain as well as diminished nerve function.

One known technique to address many such spinal conditions is commonly referred to as spinal fixation. Surgical implants are used for fusing together and/or mechanically immobilizing adjacent vertebrae of the spine. Spinal fixation may also be used to improve the position of the adjacent vertebrae relative to one another so as to alter the overall alignment of the spine. Such techniques have been used effectively to treat the above-described conditions and, in most cases, to relieve pain suffered by the patient.

One particular spinal fixation technique includes immobilizing the spine by using orthopedic rods, commonly referred to as spine rods, which run generally parallel to the spine. This is accomplished by exposing the spine posteriorly and fastening bone screws to the pedicles of the appropriate vertebrae. The pedicle screws are generally placed two per vertebra, one at each pedicle on either side of the spinous process, and serve as anchor points for the spine rods. Clamping elements adapted for receiving a spine rod there through are then used to join the spine rods to the screws. The clamping elements are commonly mounted to the head of the pedicle screws. The aligning influence of the rods forces the spine to conform to a more desirable shape. In certain instances, the spine rods may be bent to achieve the desired curvature of the spinal column.

Spinal fixation systems using orthopedic rods as described above provide a relatively rigid construct. Another type of spinal treatment technique offers dynamic stabilization for the spine. One example of a dynamic stabilization system is provided by the assignee of this invention under the trademark DYNESYS® as generally described in European Patent Application No. 0669109A1 which is hereby incorporated by reference in its entirety. Such a dynamic stabilization system utilizes pedicle screws installed into appropriate locations in adjacent vertebrae. Flexible materials in conjunction with the pedicle screws are used rather than rigid orthopedic rods or bone grafts alone as an adjunct fusion. Positioned between the pedicle screws on adjacent vertebrae is a tubular spacer with a tensioned cord passing through the central lumen of the spacer. Dynamic stabilization systems of this type bring the lumbar vertebrae into a desired anatomical position while stabilizing the effective segments.

While dynamic stabilization systems of this type offer significant advantages for spinal treatment, the proper installation and accurate position of the components in the patient's spine presents some surgeons with certain challenges. For example, the relatively high tension required to seat the tubular spacer between the adjacent pedicle screws with the cord threaded there through is an important and sometimes difficult procedure. Therefore, improved instrumentation and spinal surgery techniques for maintaining the relative position of the adjacent pedicle screws while offering a minimally invasive procedure to seat the spacer there between is desirable.

### SUMMARY OF THE INVENTION

This invention addresses these and other shortcomings in the prior art. In one embodiment, the invention is an instrument in the form of a distractor for the pedicle screw to aid surgeons in counteracting the compressive force on the spacer between the pedicle screws and assist in the process of seating the spacer. The invention also is directed to a method for seating the spacer between the pedicle screws. Most pedicle screw distractors available in the prior art are complex and bulky and require attachment to two pedicle screw heads to perform the distraction. This invention provides a simple one-piece distractor design that only requires engagement with one of the pedicle screw heads for distraction. The instrument according to this invention cooperates with existing instrumentation to distract the pedicle screw heads for simple, efficient and accurate seating of the spacer.

The pedicle screw distractor according to one embodiment includes a head specifically designed and adapted to engage one of the pedicle screws seated in the patient's spine. The head in one embodiment includes a bifurcated yoke having a concave seat adapted to receive a convex-shaped head of the pedicle screw. A lever extends from the head of the pedicle screw distractor and is joined to a handle of the instrument to form a fulcrum arrangement at the juncture between the lever and the handle. A saddle is provided at the fulcrum and is adapted to receive and seat the instrument on the shaft or body of another instrument mounted to an adjacent pedicle screw. The pedicle screw distractor when seated against the accompanying instrument and the installed pedicle screws provides for a convenient and simple distraction process by squeezing the handle toward the companion instrument thereby temporarily distracting the pedicle screws and providing access between the adjacent pedicle screws to seat the spacer of the spinal stabilization system there between.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a side elevational view of a pedicle screw distractor instrument according to one embodiment of this invention;

FIG. 2 is a rear elevational view of the instrument of FIG. 1;

FIG. 3 is an enlarged view of the head of the instrument in encircled area 3 of FIG. 2;

FIG. 4 is a cross-sectional view of the instrument head taken along line 4-4 of FIG. 3;

FIG. 5 is a side elevational view of another component of one embodiment of this invention mounted in a spinal instrument;

FIG. 6 is a perspective view of the pedicle screw distractor and companion instruments according to one embodiment of this invention in use on a patient's spine; and

FIG. 7 is an enlarged side elevational view of a portion of the instruments shown in FIG. 6.

### DETAILED DESCRIPTION OF THE INVENTION

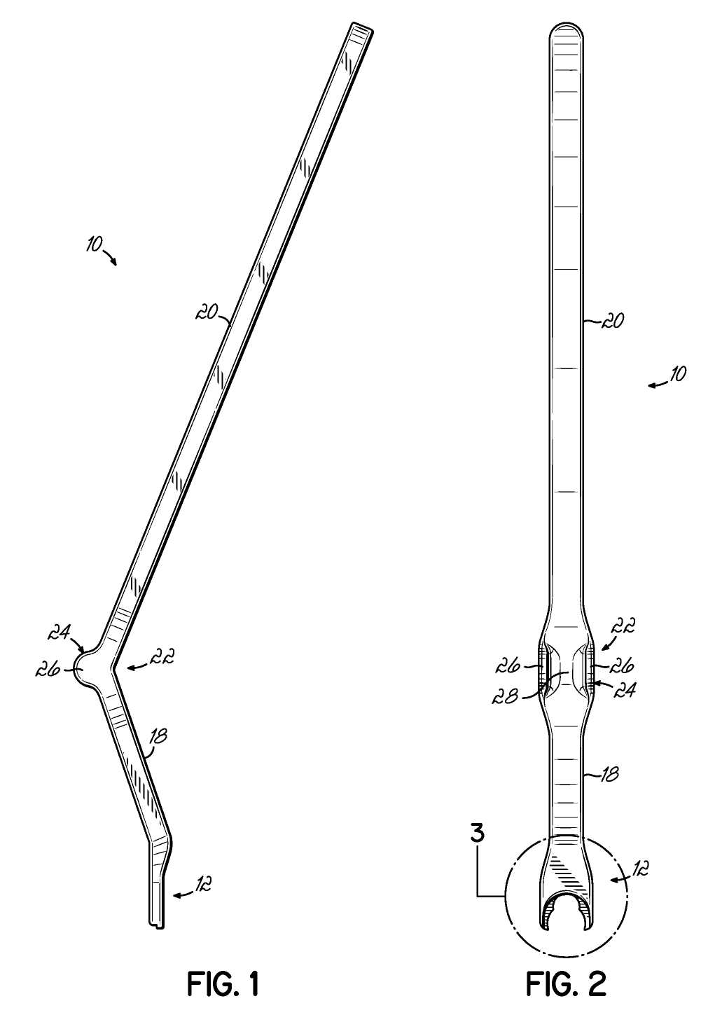

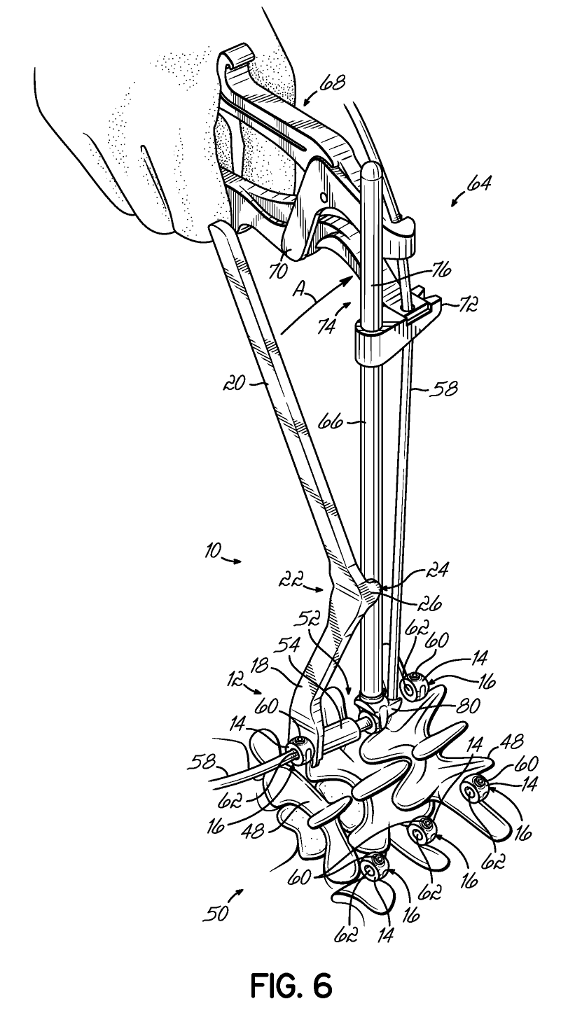

Referring to FIG. 1, one embodiment of a pedicle screw distractor 10 according to this invention is shown. The distractor 10 includes a head 12 adapted to receive a convex-shaped head 14 of a pedicle screw 16 (FIG. 6). The head 12 is joined to a lever 18 of the pedicle screw distractor 10. A handle 20 of the distractor 10 projects from a fulcrum arrangement 22 positioned at a juncture between the handle 20 and the lever 18. A saddle 24 is formed at the fulcrum 22 between the lever 18 and the handle 20 and includes a pair of spaced flanges 26 forming a channel 28 there between as shown in FIG. 2. While a variety of orientations and configurations of the pedicle screw distractor 10 are available according to this invention, a longitudinal axis of the handle 20 in one embodiment forms approximately an 138° angle with respect to a longitudinal axis of the lever 18.

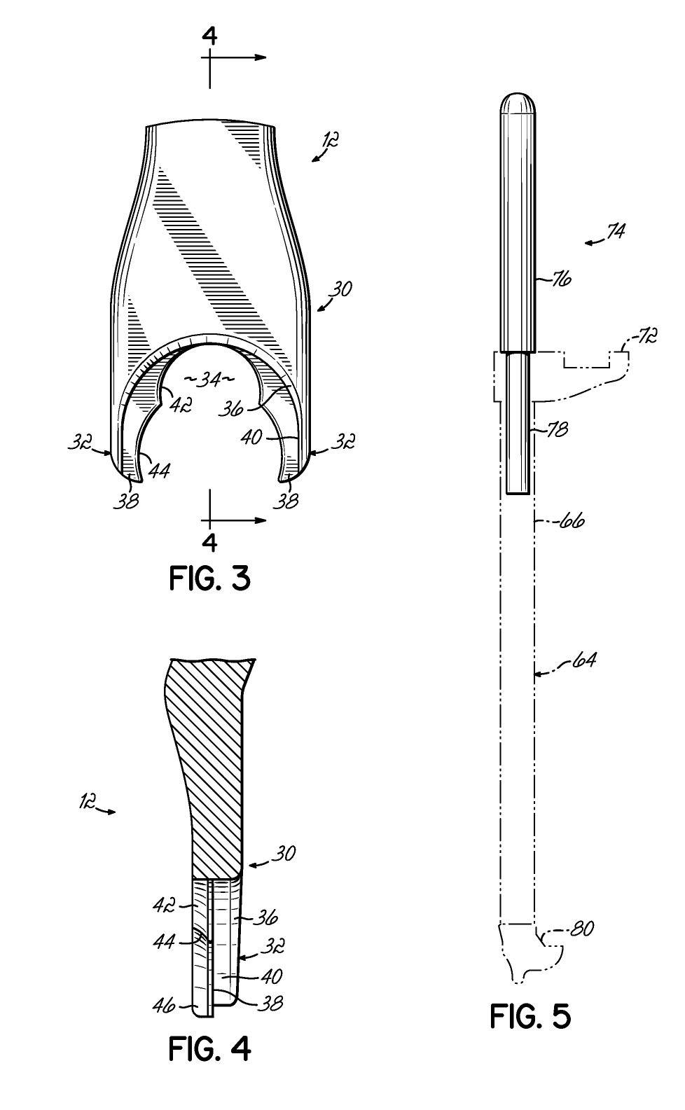

The head 12 of the distractor 10 is shown in more detail in FIGS. 2-4 and includes a bifurcated yoke 30 with a pair of spaced prongs 32 extending distally from the head 12. The spaced prongs 32 form an arcuate-shaped clearance notch 34 there between which is adapted to conveniently receive the head 14 of the pedicle screw 16 during the distraction process. The head 12 includes an arcuate-shaped forward rim 36 along a leading edge thereof. Each prong 32 includes a generally planar sill 38 in which the clearance notch 34 is formed and a midline bulk head or wall 40 is formed between the sill 38 and the forward rim 36. On a rear face of the head 12, a concave brace 42 is formed adjacent to a concave screw head seat 44. A rearward ledge 46 is formed distally from the concave seat 44 on each prong 32.

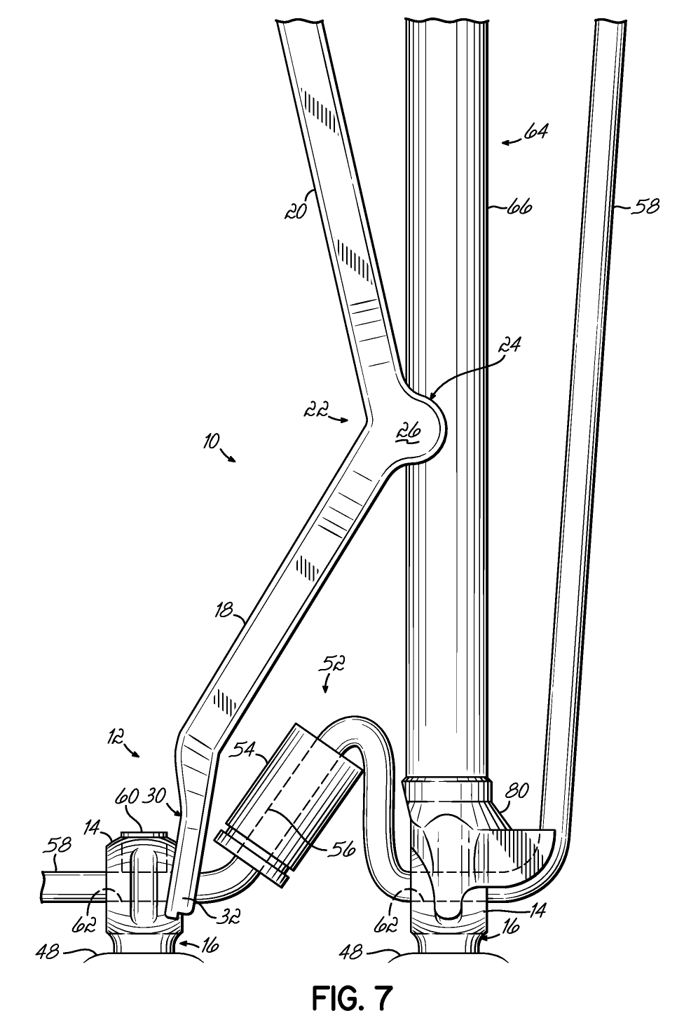

The pedicle screw distractor 10 as shown and described herein is useful in distracting the pedicle screw 16 inserted into a vertebrae 48 in a patient's spine 50 as shown in FIGS. 6 and 7. The pedicle screw 16 is part of a spinal fixation system 52 which in one embodiment is a dynamic stabilization system having a generally cylindrical spacer 54 with a central lumen 56 there through seated between adjacent pedicle screws 16 and having a cord 58 passing through the spacer 54 and the pedicle screw heads 14. According to one embodiment of such a dynamic stabilization system 52 as provided by the assignee of this invention, the spacer 54 may be a polycarbonate material and the cord 58 a polyethylene-terephalate material. The cord 58 is tensioned during the installation process between the pedicle screws 16 and secured in the tensioned orientation by a set screw 60 in each pedicle screw head 14. Each pedicle screw head 14 includes a bore 62 through which the cord 58 passes. The set screw 60 when fastened secures the tensioned cord 58 in the bore 62.

The installation procedure for the dynamic stabilization system 52 utilizes various known and existing instruments. The pedicle screw distractor 10 may be used in conjunction with a companion instrument 64 such as a DYNESYS® cord guide instrument as shown in FIGS. 6 and 7. The cord guide instrument 64 is useful to tension the cord 58 during the stabilization system 52 installation. The cord guide instrument 64 is seated on an adjacent pedicle screw 16 in the patient's spine 50 and includes an elongate shaft 66 and operates in conjunction with a cord tensioning instrument 68 for installation of the cord 58. The cord tensioning instrument 68 includes a tensioning operator 70 to be grasped by the surgeon. An arm 72 projects from the upper end of the shaft 66. A handle extension 74 may be added to the upper shaft 66 of the companion instrument 64 to provide for more convenient and stable distraction. The handle extension 74 includes an upper grip 76 and a lower post 78 which may be seated longitudinally in the shaft 66 of the instrument 64 as shown in FIG. 5.

Other companion instruments such as an anti-torque tool or the like may also be utilized in conjunction with the pedicle screw distractor 10 of this invention. Since existing instrumentation 64 is utilized in conjunction with the pedicle screw distractor 10, the companion instrument 64 such as the cord guide, anti-torque or other instrument includes a head 80 mounted on the adjacent pedicle screw 16 as shown in FIGS. 6 and 7. During the installation of the cord 58 and spacer 54, distraction of the adjacent pedicle screws 16 is advantageously accomplished with the pedicle screw distractor 10 according to this invention to facilitate the minimally invasive installation of the system 52. The concave screw head seat 44 is mounted on the pedicle screw head 14 and allows for use of the pedicle screw distractor 10 with screws 16 that are implanted in non-parallel orientations or off-axis configurations. The clearance notch 34 and screw head seat 44 provide a stable mounting of the pedicle screw distractor 10 on the pedicle screw 16. The bifurcated arrangement of the prongs 32 permits the spacer 54 to be seated against the pedicle screw 14 as well as allowing the cord 58 to pass through the screw head bore 62 without interference when the distractor 10 is seated on the screw 16. The spacer 54 sits against the sill 38 after installation and proper clearance is provided for seating of the spacer 54.

The saddle 24 is seated on the shaft 66 of the companion instrument 64 such as the cord guide, anti-torque or other tool mounted on the adjacent pedicle screw 16. Since these companion tools 64 are fixed in orientation to the pedicle screw 16 on which they are mounted, the pedicle screw distractor 10 accommodates any misalignment between the instruments 10, 64 in the saggital plane. The clearance notch 34 on the head 12 of the pedicle screw distractor 10 accommodates a range of orientations for such a purpose.

Once the pedicle screw distractor head 12 is seated on the pedicle screw 16 and the saddle 24 against the shaft 66 of the companion instrument 64, the surgeon may squeeze the handle 20 toward the companion instrument 64 in the direction of arrow A (FIG. 6) thereby distracting the pedicle screw 16 through the fulcrum arrangement 22 at the saddle 24. Once the pedicle screw distraction process is accomplished, the cord 58 may be pulled taught between the pedicle screws 16 thereby seating the spacer 54 there between. Once the spacer 54 is seated between the screws 16, the distractor 10 is removed and the pedicle screws 16 are allowed to return to their normal position for secure installation of the remainder of the spinal stabilization system 52.

From the above disclosure of the general principles of the this invention and the preceding detailed description of at least one embodiment, those skilled in the art will readily comprehend the various modifications to which this invention is susceptible. Therefore, we desire to be limited only by the scope of the following claims and equivalents thereof.

We claim:

1. A pedicle screw distractor kit comprising:
a first instrument having a head adapted to engage a first pedicle screw in a patient's spine, the first instrument having an instrument body extending from the first instrument head;
a second instrument having a head adapted to engage a second pedicle screw in the patient's spine adjacent to the first pedicle screw;
a lever extending from the head of the second instrument and having a longitudinal axis;
a handle on the second instrument spaced from the second instrument head and having a longitudinal axis; and
a fulcrum arrangement on the second instrument at a juncture between the lever and the handle;
wherein the fulcrum arrangement is adapted to engage the first instrument body when the first and second instrument heads are engaged with the respective pedicle screws and movement of the handle toward the first instrument body urges the pedicle screws away from each other.

2. The pedicle screw distractor kit of claim 1 wherein the respective axes of the handle and the lever are non-collinear.

3. The pedicle screw distractor kit of claim 1 further comprising:
a saddle proximate the fulcrum arrangement adapted to receive therein the first instrument body.

4. The pedicle screw distractor kit of claim 1 wherein the orientation of handle relative to the lever is fixed.

5. The pedicle screw distractor kit of claim 4 wherein the handle and the lever form an obtuse angle there between.

6. The pedicle screw distractor kit of claim 1 wherein the second instrument head further comprises:
a bifurcated yoke adapted to receive a head of the associated pedicle screw therein.

7. The pedicle screw distractor kit of claim 1 wherein the second instrument head further comprises:
a concave seat adapted to receive a convex shaped head of the associated pedicle screw therein.

8. The pedicle screw distractor kit of claim 1 wherein an orientation of the second instrument head is offset relative to the longitudinal axis of the lever.

9. The pedicle screw distractor kit of claim 1 further comprising:
a handle extension adapted to be selectively mounted to the first instrument body and provide a grip when the second instrument handle is being moved toward the first instrument.

10. A pedicle screw distractor kit comprising:
a first instrument having a head adapted to engage a first pedicle screw in a patient's spine, the first instrument having an instrument body extending from the first instrument head;
a second instrument having a head adapted to engage a second pedicle screw in the patient's spine adjacent to the first pedicle screw;
a bifurcated yoke having a concave seat on the second instrument head adapted to receive a convex shaped head of the associated pedicle screw therein;
a lever extending from the head of the second instrument and having a longitudinal axis;
a handle on the second instrument spaced from the second instrument head and having a longitudinal axis;
wherein the respective axes of the handle and the lever are non-collinear and form an obtuse angle there between;
a fulcrum arrangement on the second instrument at a juncture between the lever and the handle;
a saddle proximate the fulcrum arrangement adapted to receive therein the first instrument body;
wherein the saddle is adapted to engage the first instrument body when the first and second instrument heads are engaged with the respective pedicle screws and movement of the handle toward the first instrument body urges the pedicle screws away from each other.

11. The pedicle screw distractor kit of claim 10 further comprising:
a handle extension adapted to be selectively mounted to the first instrument body and provide a grip when the second instrument handle is being moved toward the first instrument.

12. A pedicle screw distractor adapted to be used in combination with a first instrument to urge apart first and second pedicle screws inserted into a patient's spine, the distractor comprising:
a distractor head adapted to engage one of the pedicle screws in the patient's spine;
a lever extending from the distractor head and having a longitudinal axis;
a handle spaced from the distractor head and having a longitudinal axis; and
a fulcrum arrangement at a juncture between the lever and the handle;
wherein the fulcrum arrangement is adapted to engage the first instrument when the distractor head is engaged with one of the pedicle screws and movement of the handle toward the first instrument urges the pedicle screws away from each other.

13. The pedicle screw distractor of claim 12 wherein the respective axes of the handle and the lever are non-collinear.

14. The pedicle screw distractor of claim 12 further comprising:
a saddle proximate the fulcrum arrangement adapted to receive therein the first instrument body.

15. The pedicle screw distractor of claim 12 wherein the orientation of the handle relative to the lever is fixed.

16. The pedicle screw distractor of claim 15 wherein the handle and the lever form an obtuse angle there between.

17. The pedicle screw distractor of claim 12 wherein the distractor head further comprises:
a bifurcated yoke adapted to receive a head of the one pedicle screws therein.

18. The pedicle screw distractor of claim 12 wherein the distractor head further comprises:
a concave seat adapted to receive a convex shaped head of the one pedicle screws therein.

19. The pedicle screw distractor of claim 12 wherein an orientation of the distractor head is offset relative to the longitudinal axis of the lever.

20. A method of distracting a pedicle screw in a patient's spine, the method comprising the steps of:
seating a head of a first instrument onto a head of the pedicle screw in the patient's spine; and
bracing a fulcrum arrangement of the first instrument against a second instrument, the fulcrum arrangement being located proximate a juncture between a handle and a lever of the first instrument, the lever being positioned between the fulcrum arrangement and the head of the first instrument;
squeezing the handle toward the second instrument to thereby pivot the lever and first instrument head about the fulcrum arrangement and distract the pedicle screw away from the second instrument.

21. The method of claim 20 further comprising:
mounting the second instrument onto a second pedicle screw in the patient's spine.

22. The method of claim 20 wherein the bracing step further comprises:
positioning a saddle proximate the fulcrum arrangement of the first instrument onto the second instrument.

23. The method of claim 20 wherein the seating step further comprises:
placing a pair of spaced prongs projecting from the head of the first instrument about the head of the pedicle screw.

24. The method of claim 20 further comprising:
coupling a handle extension to the second instrument to thereby provide a grip on the second instrument for squeezing the handle toward the second instrument.
